(19) United States
(12) Patent Application Publication
(10) Pub. No.: US 2006/0269495 A1
(43) Pub. Date: Nov. 30, 2006

(54) Alpha hydroxy acid compositions

(76) Inventors: Karl F. Popp, Schodack Landing, NY (US); Kathleen L. Clark, Medusa, NY (US)

Correspondence Address: NATH & ASSOCIATES, 112 South West Street, Alexandria, VA, 22314, US

(21) Appl. No.: 11/136,458

(22) Filed: May 25, 2005

(51) Int. Cl. A61K 8/36; A61K 31/19

(52) U.S. Cl. 424/70.1; 514/557

(57) ABSTRACT

Pharmaceutical compositions suitable for topical administration comprising an alpha hydroxy acid. In a particular aspect, these compositions exhibit improved tolerance on the skin upon topical application despite the presence of high levels of alpha hydroxy acids in the composition. These compositions are used for topical medical applications, particularly to treat various skin disorders.

### FIELD OF THE INVENTION

The present subject matter relates generally to pharmaceutical compositions suitable for topical administration comprising an alpha hydroxy acid. In a particular aspect, these compositions exhibit improved tolerance on the skin upon topical application despite the presence of high levels of alpha hydroxy acids in the composition. These compositions are used for topical medical applications, particularly to treat various skin disorders.

### BACKGROUND OF THE INVENTION

Alpha hydroxy acids are commonly known in the art as useful for treating a variety of dermatological disorders, in particular for preventing or reducing the dermatological signs of aging of the skin and/or hair, and for treating dermatological afflictions related to a disorder of the keratinization of the skin, nails and/or hair, such as acne, xerosis, ichthyosis and actinic keratosis.

For example, U.S. Pat. No. 4,105,782 discloses the treatment of acne, dandruff, and dry skin with an alpha-hydroxy acid or ammonium or organic salts thereof.

Similarly, U.S. Pat. No. 4,234,599 describes the treatment of skin keratoses with α-hydroxy acids, while U.S. Pat. No. 4,363,815 discloses the use of α-hydroxy acids for treatment of skin irritating or scalp related conditions, such as dry skin, ichthyosis, hyperkeratosis, dandruff, Darier's disease, lichen simplex chronicus, keratoses, acne, psoriasis, eczema, pruritis, warts, and herpes.

Further in this regard, U.S. Pat. No. 5,091,171 relates to methods of using combinations of alpha-hydroxy acids and amphoteric agents for skin disorders, such as wrinkles. Likewise, U.S. Pat. No. 4,424,234 relates to the use of particular alpha hydroxy carboxylic acids in aqueous solutions such as lotions, face creams, sunscreen creams, aerosol sprays, hand creams, and skin masks for treating dry skin conditions. This patent also suggests the use of such compositions to maintain skin suppleness and skin flexibility by temporary skin moisturization.

Similarly, U.S. Pat. No. 5,153,230 discloses the use of the alpha hydroxy acid glycolic acid in combination with vitamin A palmitate and vitamin E acetate for treating aging skin.

However, none of these prior compositions or methods provides a solution to the known side-effects that commonly appear when an alpha-hydroxy acid is topically administered to a patient, particularly in a high amount. These alpha-hydroxy acid side-effects can include stinging, tingling, itching, or burning sensations, as well as redness and peeling, which can result in considerable discomfort. Further, alpha-hydroxy acids are known to irritate human skin on repeated topical applications.

Further, the prior art typically does not disclose or suggest storage-stable compositions containing an alpha hydroxy acid that does not exhibit degradation over time. In this regard, topical compositions are stored at a controlled room temperature of about 15 to about 30° C. However, previous lactic acid products, for example, stored at this temperature at times exhibit significant degradation of the lactic acid. Further, it is often difficult even to produce a topical composition containing substantial quantities of lactic acid, as these compositions can be unstable and difficult to form.

In fact, it is acknowledged in the prior art that large amounts of α-hydroxy acids are difficult to formulate in topical dosage forms, such as a cream emulsion. In this regard, U.S. Pat. No. 6,074,653 discloses that when incorporated into topical formulations in a concentrated amount, α-hydroxy acids render the formulations unstable and therefore difficult to commercially exploit.

U.S. Pat. No. 4,772,592 further recognizes the difficulties in forming stable alpha hydroxy acid compositions. This patent describes a stable water-in-oil emulsion for treating acne, which includes a C₁-C₄ alkyl lactate (alpha hydroxy acid), a silicone oil, a nonionic liquid emulsifier, and a C₁-C₄ alkanol in specific amounts. Volatile polar liquids such as the alkanols are essential ingredients of this emulsion because, together with the silicone oil, they stabilize the emulsion. However, the alkanols present the disadvantage of being irritants to the skin or to the mucosa and hence of contributing to the aforesaid discomfort caused by the alpha hydroxy acids.

In view of these difficulties, many of the presently existing alpha hydroxy acid products contain less than an optimal amount of the alpha hydroxy acid required to maximize their effectiveness in treating the dermatological disorder at issue. These lesser amounts of the alpha hydroxy acid are typically used to minimize the potential side effects and formulation stability difficulties discussed above.

Accordingly, there remains a need in the art for topical compositions containing a large amount of an alpha-hydroxy acid useful in treating a variety of dermatological disorders that do not produce stinging, tingling, itching, or burning sensations upon topical administration to a patient. Further, there remains a need for topical compositions containing a large amount of an alpha-hydroxy acid that are storage stable and do not exhibit significant degradation over time when stored at a controlled room temperature. The present subject matter addresses these needs.

### SUMMARY OF THE INVENTION

The present subject matter relates generally to a storage-stable pharmaceutical composition comprising high levels of an alpha hydroxy acid that exhibits improved tolerance on skin and methods of using the same to treat various skin disorders.

In this regard, a preferred embodiment of the present subject matter relates to a storage-stable pharmaceutical composition providing improved tolerance upon topical administration comprising:

a) about 10 to about 45% by weight of an alpha hydroxy acid or a pharmaceutically acceptable salt thereof as an active ingredient;

b) a petroleum derivative;

c) a silicone containing emulsifier;

d) water; and

e) remaining amounts of one or more dermatologically acceptable excipients;

wherein said petroleum derivative and said silicone containing emulsifier are present in a ratio sufficient to render said composition storage-stable and well tolerated upon topical administration.

Another preferred embodiment of the present subject matter relates to a method of treating a skin disorder in a mammal, comprising topically administering to skin of a mammal in need thereof a therapeutically effective amount of a storage stable pharmaceutical composition providing improved tolerance upon topical administration comprising:

a) about 10 to about 45% by weight of an alpha hydroxy acid or a pharmaceutically acceptable salt thereof as an active ingredient;

b) a petroleum derivative;

c) a silicone containing emulsifier;

d) water; and

e) remaining amounts of one or more dermatologically acceptable excipients;

wherein said petroleum derivative and said silicone containing emulsifier are present in a ratio sufficient to render said composition well tolerated upon topical administration.

Yet another preferred embodiment of the present subject matter relates to a method of treating keratosis pilaris in a mammal, comprising topically administering to skin of a mammal in need thereof a therapeutically effective amount of an improved tolerance, storage-stable pharmaceutical composition suitable for topical administration comprising about 10 to about 45% by weight of an alpha hydroxy acid or a pharmaceutically acceptable salt thereof as an active ingredient.

Still another preferred embodiment of the present subject matter relates to a storage-stable pharmaceutical composition providing improved tolerance upon topical administration comprising:

a) about 10 to about 45% by weight of lactic acid or a pharmaceutically acceptable salt thereof as an active ingredient;

b) a petroleum derivative that is mineral oil;

c) a silicone containing emulsifier that is a polysiloxane;

d) water; and

e) remaining amounts of one or more dermatologically acceptable excipients;

wherein said petroleum derivative and said silicone containing emulsifier are present in a weight ratio of about 0.75:1 to about 1:0.75 sufficient to provide a composition of improved tolerance upon topical administration.

Still yet another preferred embodiment of the present subject matter relates to a method of treating a skin disorder in a mammal, comprising topically administering to skin of a mammal in need thereof a therapeutically effective amount of a storage-stable pharmaceutical composition providing improved tolerance upon topical administration comprising:

a) about 10 to about 45% by weight of lactic acid or a pharmaceutically acceptable salt thereof as an active ingredient;

b) a petroleum derivative that is mineral oil;

c) a silicone containing emulsifier that is a polysiloxane;

d) water; and

e) remaining amounts of one or more dermatologically acceptable excipients;

wherein said petroleum derivative and said silicone containing emulsifier are present in a weight ratio of about 0.75:1 to about 1:0.75 sufficient to provide a composition of improved tolerance upon topical administration.

A further preferred embodiment of the present subject matter relates to a process for preparing a storage-stable pharmaceutical composition providing improved tolerance upon topical administration, said process comprising:
1) preparing an aqueous phase comprising about 10 to about 45% by weight of the overall weight of the composition of an alpha hydroxy acid or a pharmaceutically acceptable salt thereof as an active ingredient, about 0.005 to about 0.5% by weight of the overall weight of the composition of a chelating agent, and water at a temperature of about 55 to about 83° C.;
2) preparing an oil phase comprising a petroleum derivative and a silicone containing emulsifier in a weight ratio of about 0.75:1 to about 1:0.75, and about 0.02 to about 0.225% by weight of the overall weight of the composition of an antioxidant at a temperature of about 55 to about 85° C.;
3) adding said oil phase to said aqueous phase while mixing at a temperature of about 55 to about 83° C. to obtain a homogenous emulsion;
4) cooling said emulsion to a temperature of about 15 to about 30° C.; and
5) recovering a storage-stable, well tolerated topical pharmaceutical composition.

### DETAILED DESCRIPTION OF THE INVENTION

### Definitions

As used herein, the terms “alpha hydroxy acid”, “alpha-hydroxy acid”, and “α-hydroxy acid” all mean and refer to the same type of acid, e.g., organic carboxylic acids in which one hydroxyl group is attached to the alpha carbon of the acids.

As used herein, an “extended period of time” refers to the shelf life of the presently preferred compositions, including time spent on the shelf at a pharmacy as well as the entire time period after sale of the composition during which the composition remains effective for the indicated use.

As used herein, “improved tolerance” or “well tolerated” refers to the ability of the presently preferred compositions to reduce any of the known side-effects that commonly appear when an alpha-hydroxy acid is topically administered to a patient, including stinging, tingling, itching, or burning sensations, as well as redness and peeling.

As used herein, “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids.

Non-limiting examples of base salts include ammonium salts; alkali metal salts, such as sodium and potassium salts; alkaline earth metal salts, such as calcium and magnesium salts; salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, “storage stable” refers to the ability of the present compositions to have a long shelf life, including time spent on the shelf at a pharmacy as well as the entire time period after sale of the composition, during which time the composition maintains its effectiveness and pharmaceutically acceptable appearance. Accordingly, the present compositions are stable in that they exhibit a minimum amount of degradation during an extended period of storage.

Other terms as used herein are meant to be defined by their well-known meanings in the art.

### Topical Pharmaceutical Compositions

A preferred aspect of the subject matter expressed herein relates to storage-stable pharmaceutical compositions providing improved tolerance upon topical administration comprising:

a) about 10 to about 45% by weight of an alpha hydroxy acid or a pharmaceutically acceptable salt thereof as an active ingredient;

b) a petroleum derivative;

c) a silicone containing emulsifier;

d) water; and

e) remaining amounts of one or more dermatologically acceptable excipients;

wherein said petroleum derivative and said silicone containing emulsifier are present in a ratio sufficient to render said composition storage-stable and well tolerated upon topical administration.

In a preferred embodiment, these pharmaceutical compositions comprise about 30 to about 80% by weight water.

The preferred pharmaceutical compositions described herein are unique in that they are well tolerated upon topical application to a patient despite the presence of high levels of the alpha hydroxy acid active ingredient. Accordingly, these compositions are advantageous over previous compositions that contain lower levels of the alpha hydroxy acid active ingredient in order to minimize the stinging, tingling, itching, or burning sensations, as well as redness and peeling, normally associated with alpha hydroxy acids. Despite the use of these lower levels, many of the previous compositions still exhibit some of these side effects upon topical application.

As such, it would be expected that the present compositions would exhibit an increased patient compliance in comparison with alpha hydroxy acid compositions previously known in the art due to the reduced incidence of side effects. Further, the present compositions should be more effective in treating various dermatological disorders than the previously known compositions due to the presence of higher amounts of the alpha hydroxy acid active agent.

The reduced incidence of side effects of alpha-hydroxy acids is, in part, attributable to the increased stability of the presently preferred compositions. In this regard, the preferred topical pharmaceutical compositions are further unique in that they are storage stable with respect to the alpha hydroxy acid active ingredient. Accordingly, these compositions have a decided advantage over previous alpha hydroxy acid compositions in that they limit the amount of degradation of the active ingredient over time, resulting in a composition with improved long-term efficacy at temperatures of about 30° C. or below. In this regard, the present compositions are preferably able to maintain a purity of at least 90% and a concentration of degradation product(s) less than about 10% of the starting concentration of the alpha hydroxy acid.

The stability of the preferred compositions also provides an enhanced skin-feel to a patient being treated. This is another reason why it would be expected that these compositions would result in an increased patient compliance with the strict daily regimen of topical alpha hydroxy acid administration.

Further, the remarkable stability of the preferred compositions solves long felt difficulties in formulating alpha hydroxy acid compositions. Since these compositions have an increased stability over alpha hydroxy acid compositions previously known in the art, they provide unexpected advantages over the prior art compositions. For example, the increased storage stability permits the presently preferred compositions to be manufactured in greater quantities without fear that the compositions produced will be wasted. Further, the enhanced stability provides the presently preferred compositions with an enhanced effect in treating skin disorders treatable with alpha hydroxy acids over the previously known compositions.

The selection of specific excipients and amounts thereof in the presently preferred compositions, as well as the preparation of compositions having a specific designated pH in the form of a designated emulsion, conveys these unique stability and improved tolerance characteristics to the presently preferred compositions. In particular, the presence of a petroleum derivative and a silicone containing emulsifier in a particular weight ratio enhances the stability of the presently preferred compositions, and aids in rendering these compositions well tolerated upon topical administration.

In this regard, the present compositions preferably contain a petroleum derivative and a silicone containing emulsifier in a weight ratio of about 0.75:1 to about 1:0.75. In a particularly preferred embodiment, the weight ratio of these two components is about 0.9:1 to about 1:0.9. These particular weight ratios are narrowly tailored to maximize the stability and improved tolerance characteristics of the preferred compositions described herein.

Moreover, by virtue of the specific formulations enumerated herein, the release and/or absorption of the active alpha-hydroxy acid from the preferred compositions may be attained slowly and gradually when the composition is topically applied to the skin, nails, hair, and/or the scalp, if desired, which can make it very pleasing for use by a patient.

Preferred compositions are formed as an oil-in-water emulsion, i.e. an emulsion having an oil phase and an aqueous phase. Preferably, formation of the emulsion is aided by the silicone containing emulsifier. More preferably, the emulsion is formed using at least one additional emulsifier.

The α-hydroxy acid or pharmaceutically acceptable salt thereof active ingredient is preferably contained in the aqueous phase of the emulsion. The pH of this aqueous phase, if required, is adjusted to a range of about 2 to about 8. Since the emulsion is an oil-in-water emulsion having water as the major component, the final composition will have a pH mirroring that of the aqueous phase. Accordingly, the pH of the final composition preferably ranges from about 2 to about 8. In a more preferred embodiment, the pH of both the aqueous phase and the final composition ranges from about 2 to about 6. In a particularly preferred embodiment, the pH of both the aqueous phase and the final composition ranges from about 2.5 to about 4.

These particular emulsion and pH characteristics provide the unique stability and improved tolerance advantages of the presently preferred compositions. These characteristics permit these compositions to exhibit a longer shelf life, increased pharmaceutical effectiveness, and decreased incidence of side effects when compared with other alpha hydroxy acid products previously known in the art.

α-Hydroxy Acids

The present compositions preferably comprise about 10 to about 45% by weight of an α-hydroxy acid or a pharmaceutically acceptable salt thereof as an active ingredient. In a particularly preferred embodiment, these compositions comprise about 21 to about 40% by weight of the α-hydroxy acid or a pharmaceutically acceptable salt thereof. In a most preferred embodiment, the present compositions comprise about 30% by weight of pure α-hydroxy acid in free acid form.

In this regard, a person of ordinary skill in the art would understand that the content of the α-hydroxy acid is based on the purity of the material used. For example, a presently preferred composition that contains lactic acid USP in an amount of about 34.09% by weight actually contains approximately 30% by weight of lactic acid.

The α-hydroxy acids present in the compositions described herein may be present in any pure isomeric form, or in a racemic mixture of these isomeric forms.

The α-hydroxy acids useful in the preferred compositions are organic carboxylic acids in which one hydroxyl group is attached to the alpha carbon of the acids. The generic structure of such alpha hydroxyacids may be represented as follows:
(Rₐ)(R_{b})C(OH)COOH
where Rₐ and R_{b} each independently are an H, F, Cl, Br, alkyl, aralkyl, or aryl group of saturated or unsaturated, isomeric or non-isomeric, straight or branched chain or cyclic form, having 1 to 25 carbon atoms. In addition Rₐ and R_{b} may each carry one or more OH, CHO, COOH, or alkoxy groups having 1 to 9 carbon atoms. The α-hydroxy acids may exist as stereoisomers as D, L, and DL forms when Rₐ and R_{b} are not identical.

The α-hydroxy acid may be present in the preferred compositions as a free acid, in lactone form, or in a salt form with an organic base or an inorganic alkali. In a preferred embodiment, the α-hydroxy acid is present as a mixture of an acid and a salt.

Typical alkyl, aralkyl and aryl groups for Rₐ and R_{b} include methyl, ethyl, propyl, isopropyl, butyl, pentyl, octyl, lauryl, stearyl, benzyl, and phenyl, etc. These α-hydroxy acids may be divided into the following non-limiting exemplary groups: (1) alkyl α-hydroxy acids, (2) aralkyl and aryl α-hydroxy acids, (3) polyhydroxy α-hydroxy acids, and (4) polycarboxylic α-hydroxy acids. The following are representative, non-limiting examples of α-hydroxy acids in each subgroup.

(1) Alkyl α-Hydroxy Acids

1. 2-Hydroxyethanoic acid (Glycolic acid, hydroxyacetic acid)
   (H)(H)C(OH)COOH
2. 2-Hydroxypropanoic acid (Lactic acid)
   (CH₃)(H)C(OH)COOH
3. 2-Methyl 2-hydroxypropanoic acid (Methyllactic acid)
   (CH₃)(CH₃)C(OH)COOH
4. 2-Hydroxybutanoic acid
   (C₂H₅)(H)C(OH)COOH
5. 2-Hydroxypentanoic acid
   (C₃H₇)(H)C(OH)COOH
6. 2-Hydroxyhexanoic acid
   (C₄H₉)(H)C(OH)COOH
7. 2-Hydroxyheptanoic acid
   (C₅H₁₁)(H)C(OH)COOH
8. 2-Hydroxyoctanoic acid
   (C₆H₁₃)(H)C(OH)COOH
9. 2-Hydroxynonanoic acid
   (C₇H₁₅)(H)C(OH)COOH
10. 2-Hydroxydecanoic acid
   (C₈H₁₇)(H)C(OH)COOH
11. 2-Hydroxyundecanoic acid
   (C₉H₁₉)(H)C(OH)COOH
12. 2-Hydroxydodecanoic acid (Alpha hydroxylauric acid)
   (C₁₀H₂₁)(H)C(OH)COOH
13. 2-Hydroxytetradecanoic acid (Alpha hydroxymyristic acid)
   (C₁₂H₂₅)(H)C(OH)COOH
14. 2-Hydroxyhexadecanoic acid (Alpha hydroxypalmitic acid)
   (C₁₄H₂₉)(H)C(OH)COOH
15. 2-Hydroxyoctadecanoic acid (Alpha hydroxystearic acid)
   (C₁₆H₃₄)(H)C(OH)COOH
16. 2-Hydroxyeicosanoic acid (Alpha hydroxyarachidonic acid)
   (C₁₈H₃₇)(H)C(OH)COOH
   (2) Aralkyl and Aryl α-Hydroxy Acids
1. 2-Phenyl 2-hydroxyethanoic acid (Mandelic acid)
   (C₆H₅)(H)C(OH)COOH
2. 2,2-Diphenyl 2-hydroxyethanoic acid (Benzilic acid)
   (C₆H₅)(C₆H₅)C(OH)COOH
3. 3-Phenyl 2-hydroxypropanoic acid (Phenyllactic acid)
   (C₆H₅CH₂)(H)C(OH)COOH
4. 2-Phenyl 2-methyl 2-hydroxyethanoic acid (Atrolactic acid)
   (C₆H₅)(CH₃)C(OH)COOH
5. 2-(4′-Hydroxyphenyl) 2-hydroxyethanoic acid (4-Hydroxymandelic acid)
   (HO—C₆H₄)(H)C(OH)COOH
6. 2-(4′-Chlorophenyl) 2-hydroxyethanoic acid (4-Chloromandelic acid)
   (Cl—C₆H₄)(H)C(OH)COOH
7. 2-(3′-Hydroxy-4′-methoxyphenyl) 2-hydroxyethanoic acid (3-Hydroxy-4-methoxymandelic acid)
   (HO—,CH₃O—C₆H₃)(H)C(OH)COOH
8. 2-(4′-Hydroxy-3′-methoxyphenyl) 2-hydroxyethanoic acid (4-Hydroxy-3-methoxymandelic acid)
   (HO—,CH₃O—C₆H₃)(H)C(OH)COOH
9. 3-(2′-Hydroxyphenyl) 2-hydroxypropanoic acid [3-(2′-Hydroxyphenyl)lactic acid]
   HO—C₆H₄—CH₂(H)C(OH)COOH
10. 3-(4′-Hydroxyphenyl) 2-hydroxypropanoic acid [3-(4′-Hydroxyphenyl)lactic acid]
   HO—C₆H₄—CH₂ (H)C(OH)COOH
11. 2-(3′,4′-Dihydroxyphenyl) 2-hydroxyethanoic acid (3,4-Dihydroxymandelic acid)
   HO—,HO—C₆H₃(H)C(OH)COOH
   (3) Polyhydroxy α-Hydroxy Acids
1. 2,3-Dihydroxypropanoic acid (Glyceric acid)
   (HOCH₂)(H)C(OH)COOH
2. 2,3,4-Trihydroxybutanoic acid (Isomers; erythronic acid, threonic acid)
   HOCH₂(HO)CH₂(H)C(OH)COOH
3. 2,3,4,5-Tetrahydroxypentanoic acid (Isomers; ribonic acid, arabinoic acid, xylonic acid, lyxonic acid)
   HOCH₂(HO)CH₂(HO)CH₂(H)C(OH)COOH
4. 2,3,4,5,6-Pentahydroxyhexanoic acid (Isomers; allonic acid, altronic acid, gluconic acid, mannoic acid, gulonic acid, idonic acid, galactonic acid, talonic acid)
   HOCH₂(HO)CH₂(HO)CH₂(HO)CH₂(H)C(OH)COOH
5. 2,3,4,5,6,7-Hexahydroxyheptanoic acid (Isomers; glucoheptonic acid, galactoheptonic acid etc.)
   HOCH₂(HO)CH₂(HO)CH₂(HO)CH₂(HO)CH₂(H)C(OH)COOH
   (4) Polycarboxylic α-Hydroxy Acids
1. 2-Hydroxypropane-1,3-dioic acid (Tartronic acid)
   HOOC(H)C(OH)COOH
2. 2-Hydroxybutane-1,4-dioic acid (Malic acid)
   HOOCCH₂(H)C(OH)COOH
3. 2,3-Dihydroxybutane-1,4-dioic acid (Tartaric acid)
   HOOC(HO)CH(H)C(OH)COOH
4. 2-Hydroxy-2-carboxypentane-1,5-dioic acid (Citric acid)
   HOOCCH₂C(OH)(COOH)CH₂COOH
5. 2,3,4,5-Tetrahydroxyhexane-1,6-dioic acid (Isomers; saccharic acid, mucic acid etc.)
   HOOC(CHOH)₄COOH

Particularly preferred α-hydroxy acids useful in the present compositions are those selected from the group consisting of atrolactic acid, benzilic acid, 4-chloromandelic acid, citric acid, 3,4-dihydroxymandelic acid, ethyl pyruvate, galacturonic acid, gluconolactone, glucuronic acid, glucuronolactone, glycolic acid, 2-hydroxybutanoic acid, 2-hydroxypentanoic acid, 2-hydroxyhexanoic acid, 2-hydroxyheptanoic acid, 2-hydroxyactanoic acid, 2-hydroxynonanoic acid, 2-hydroxydecanoic acid, 2-hydroxyundecanoic acid, 4-hydroxymandelic acid, 3-hydroxy-4-methoxymandelic acid, 4-hydroxy-3-methoxymandelic acid, α-hydroxyarachidonic acid, α-hydroxybutyric acid, α-hydroxyisobutyric acid, α-hydroxylauric acid, α-hydroxymyristic acid, α-hydroxypalmitic acid, α-hydroxystearic acid, 3-(2′-hydroxyphenyl)lactic acid, 3-(4′-hydroxyphenyl)lactic acid, lactic acid, malic acid, mandelic acid, methyllactic acid, methylpyruvate, mucic acid, α-phenylactic acid, α-phenylpyruvic acid, pyruvic acid, saccharic acid, tartaric acid, tartronic acid, pharmaceutically acceptable salts thereof, derivatives thereof, and mixtures thereof.

In a most preferred embodiment, the α-hydroxy acid is lactic acid or a pharmaceutically acceptable salt thereof.

Petroleum Derivative

The preferred compositions further comprise a petroleum derivative as an essential component. In this regard, the petroleum derivative is preferably present, in conjunction with the silicone containing emulsifier, in a ratio sufficient to impart the unique stability and improved tolerance characteristics to the present preferred composition. Accordingly, in a preferred embodiment, the petroleum derivative is present with the silicone containing emulsifier in a weight ratio of about 0.75:1 to about 1:0.75. In a particularly preferred embodiment, the petroleum derivative is present in conjunction with the silicone containing emulsifier in a weight ratio of about 0.9:1 to about 1:0.9.

In another preferred embodiment, the petroleum derivative is present in the instant compositions in an amount of about 3 to about 13% by weight. In a particularly preferred embodiment, these compositions comprise about 3.75 to about 10% by weight of the petroleum derivative.

In a preferred embodiment, non-limiting examples of petroleum derivatives useful in this regard include a paraffin, a mineral oil, petrolatum, a synthetic or semi-synthetic hydrocarbon, derivatives thereof, and mixtures thereof.

Particularly preferred, non-limiting examples of paraffins useful in the present compositions include liquid paraffin, light liquid paraffin, paraffin oil, hard paraffin, paraffin alcohols, paraffin wax, synthetic paraffin, derivatives thereof, and mixtures thereof.

Particularly preferred, non-limiting examples of mineral oils useful in the present compositions include heavy mineral oil, light mineral oil, white mineral oil, mineral oil alcohols, derivatives thereof, and mixtures thereof.

Particularly preferred, non-limiting examples of petrolatum useful in the present compositions include liquid petrolatum, white petrolatum, a semi-solid petrolatum, derivatives thereof, and mixtures thereof.

Silicone Containing Emulsifier

The preferred compositions further comprise a silicone containing emulsifier as an essential component. This silicone containing emulsifier further helps maintain the storage stability and improved tolerance effects of these preferred compositions, particularly when present with the petroleum derivative in the weight ratio specified above. In a preferred embodiment, the silicone containing emulsifier is present in the instant compositions in an amount of about 4 to about 10% by weight. In a particularly preferred embodiment, these compositions comprise about 5 to about 8% by weight of the silicone containing emulsifier.

In a preferred embodiment, the silicon containing emulsifier useful herein is a polysiloxane or a derivative thereof.

Particularly preferred, non-limiting examples of polysiloxanes useful in the present compositions include cyclomethicone, dimethicone, simethicone, pharmaceutically acceptable salts thereof, derivatives thereof, and mixtures thereof.

Additional Emulsifiers

In a preferred embodiment, the present compositions may further comprise an additional emulsifier. In this regard, the preferred compositions herein may contain up to about 12% by weight of an additional emulsifier. The presence of this additional emulsifier in the specified total amount may further contribute to the improved tolerance, storage stable characteristics of the presently preferred compositions.

Preferred, non-limiting examples of such additional emulsifiers useful herein include straight or branched chain fatty acids, polyoxyethylene sorbitan fatty acid esters, lecithin, sorbitan fatty acid esters, propylene glycol stearate, glyceryl stearate, polyethylene glycol, fatty alcohols, ethoxylated fatty alcohols, fatty alcohol esters, fatty alcohol ethers, polymeric ethylene oxide-propylene oxide block polymers, derivatives thereof, pharmaceutically acceptable salts thereof, and mixtures thereof. In this regard, preferred exemplary ethoxylated fatty alcohols useful herein have a degree of ethoxylation from 0 to 30.

Particularly preferred, non-limiting examples of additional emulsifiers in this regard are selected from the group consisting of steareth-2, steareth-21, ceteareth-20, cetostearyl alcohol, derivatives thereof, and mixtures thereof.

Dermatologically Acceptable Excipients

The preferred compositions discussed herein additionally comprise remaining amounts of one or more dermatologically acceptable excipients. Preferred, non-limiting examples of dermatologically acceptable excipients useful in these compositions are those selected from the group consisting of surfactants, preservatives, emollients, humectants, fluid alkyl alcohols, thickening agents, suspending agents, pH modifiers/buffering agents, chelating agents, antioxidants, derivatives thereof, and mixtures thereof.

Surfactants

The presently preferred compositions may optionally contain one or more surfactants as a dermatologically acceptable excipient. Non-limiting examples of surfactants that can optionally be included in these preferred compositions include nonionic surfactants, anionic surfactants, amphoteric surfactants, zwitterionic surfactants, cationic surfactants, derivatives thereof, and mixtures thereof.

Preservatives

The presently preferred compositions may optionally further contain a preservative. Preferred non-limiting examples of preservatives that can optionally be included in these compositions include methylparaben, benzalkonium chloride, propylparaben, benzoic acid, EDTA, phenolic acid, sorbic acid, benzyl alcohol, isopropyl alcohol, benzethonium chloride, bronopol, butylparaben, cetrimide, chlorhexidine, chlorobutanol, chlorocresol, cresol, ethylparaben, glycerol, imidurea, phenol, phenoxyethanol, phenylethyl alcohol, postassium sorbate, propylene glycol, sodium benzoate, sodium propionate, sorbic acid, thimerosol, derivatives thereof, and mixtures thereof.

Particularly preferred preservatives in this regard are selected from the group consisting of propylparaben, methylparaben, derivatives thereof, and mixtures thereof.

Emollients

The presently preferred compositions may optionally further contain an emollient. Preferred non-limiting examples of emollients that can optionally be included in these compositions include myristyl lactate, isopropyl palmitate, cetearyl alcohol, lanolin, ceryl esters wax, cholesterol, phytosterols, glycerol, glycerol monostearate, isopropyl myristate, lecithin, derivatives thereof, and mixtures thereof.

Humectants

The presently preferred compositions may optionally further contain a humectant or a moisturizer. Preferred non-limiting examples of humectants and/or moisturizers that can optionally be included in these compositions include glycerin, propylene glycol, sorbitol, triacetin, derivatives thereof, and mixtures thereof.

Fluid Alkyl Alcohols

The presently preferred compositions may optionally further contain a fluid alkyl alcohol. Preferred non-limiting examples of fluid alkyl alcohols that can optionally be included in these compositions include ethanol, isopropyl alcohol, octodecyl alcohol, propyl alcohol, butanol, pentanol, derivatives thereof, and mixtures thereof.

A particularly preferred fluid alkyl alcohol in this regard is ethanol or a derivative thereof.

Thickening Agents

The presently preferred compositions may optionally further contain a thickening agent. Preferred non-limiting examples of thickening agents that can optionally be included in these compositions include cetyl alcohol, Carbomers, acrylates/C10-30 alkyl acrylate crosspolymers, hydroxyethylcellulose, hydroxypropylcellulose, polyethylene oxide, alginic acid, bentonite, carboxymethylcellulose and salts thereof, microcrystalline cellulose, colloidal silicon dioxide, gelatin, guar gum, xanthan gum, magnesium aluminum silicate, maltitol, methylcellulose, polyoxyethylene fatty acid esters, polyvinylpyrrolidone, propylene glycol alginate, sodium alginate, sorbitan fatty acid esters, tragacanth, derivatives thereof, and mixtures thereof.

A particularly preferred thickening agent in this regard is xanthan gum or a derivative thereof.

pH Modifiers/Buffering Agent

The presently preferred compositions may optionally contain a sufficient amount of a buffering agent, or a pH modifier, to provide an overall pH of about 2 to about 6 to said compositions.

Preferred buffering agents in this regard are selected from the group consisting of pharmaceutically acceptable acids, bases, and mixtures thereof. Preferred non-limiting examples of buffering agents in this regard include acetic acid, acetylsalicyclic acid, ascorbic acid, boric acid, carbonic acid, citric acid, formic acid, ethanesulfonic acid, fumaric acid, glycerophosphoric acid, hippuric acid, hydrochloric acid, maleic acid, methanesulfonic acid, nitrous acid, oxalic acid, phosphoric acid, saccharin, sorbic acid, sulfuric acid, thiosulfuric acid, undecylenic acid, ethanolamine, triethanolamine, sodium carbonate, sodium acetate, sodium hydrogen phosphate, sodium dihydrogen phosphate, sodium citrate, sodium bicarbonate, sodium hydroxide, derivatives thereof, and mixtures thereof.

Particularly preferred buffering agents in this regard are selected from the group consisting of citric acid, phosphoric acid, sodium hydroxide, derivatives thereof, and mixtures thereof.

Chelating Agents

The presently preferred compositions may optionally further contain a chelating agent. Preferred non-limiting examples of chelating agents that can optionally be included in these compositions include citric acid, isopropyl (mono) citrate, stearyl citrate, lecithin citrate, gluconic acid, tartaric acid, oxalic acid, phosphoric acid, sodium tetrapyrophosphate, potassium monophosphate, sodium hexametaphosphate, calcium hexametaphosphate, sorbitol, glycine (aminoacetic acid), methyl glucamine, triethanolamine (trolamine), EDTA, DEG (dihydroxyethylglycine), DPTA (diethylene triamine pentaacetic acid), NTA (Nitrilotriacetic Acid), HEDTA (N-(hydroxyethyl)-ethylenetriaminetriacetic acid), aminocarboxylates, dimercaperol (BAL), larixinic acid (Maltol), unidentate ligands (fluoride and cyanide ions), diphenylthiocarbazone, 0-phenanthroline, barium diphenylamine sulfonate, sodium glucoheptonate, 8-hydroxyquinoline, olefin complexes (such as dicyclopentadienyl iron), porphyrins, phosponates, pharmaceutically acceptable salts thereof, derivatives thereof, and mixtures thereof.

Particularly preferred chelating agents in this regard are selected from the group consisting of EDTA, edetate disodium, citric acid, pharmaceutically acceptable salts thereof, derivatives thereof, and mixtures thereof.

Preferred, non-limiting examples of pharmaceutically acceptable salts or derivatives of EDTA useful in the preferred compositions include edetate disodium, trans-1,2-diaminocyclohexane-N,N,N′,N′-tetraaceticacid monohydrate, N,N-bis(2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′,N′-tetrakis(methylenephosponic acid), 0,0′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N′,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11]pentatriacontane hexahydrobromide, triethylenetetramine-N,N,N′,N″, N′″,N′″-hexaacetic acid, pharmaceutically acceptable salts thereof, derivatives thereof, and mixtures thereof.

Antioxidants

The presently preferred compositions may optionally further contain an antioxidant. Preferred non-limiting examples of antioxidants that can optionally be included in these compositions include butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and mixtures thereof. Particularly preferred antioxidants in this regard are selected from the group consisting of butylated hydroxytoluene, alpha tocopherol, pharmaceutically acceptable salts thereof, derivatives thereof, and mixtures thereof.

In addition to those enumerated above, any other petroleum derivative, silicone containing emulsifier, additional emulsifier, surfactant, preservative, emollient, humectant, fluid alkyl alcohol, thickening agent, suspending agent, pH modifier, chelating agent, antioxidant, or other dermatologically acceptable excipient commonly known to those of ordinary skill in the art as useful in topical compositions is contemplated as useful in the compositions described herein. Further, any non-toxic, inert, and effective topical carrier may be used to formulate the compositions described herein. Well-known carriers used to formulate other topical therapeutic compositions for administration to humans will be useful in these compositions. Examples of these components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety. Examples of such useful pharmaceutically acceptable excipients, carriers and diluents include distilled water, physiological saline, Ringer's solution, dextrose solution, Hank's solution, and DMSO, which are among those preferred for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

In another particularly preferred embodiment, the presently preferred pharmaceutical compositions are formulated in a lotion, cream, ointment, shampoo, gel, aerosol, or other pharmaceutically acceptable topical dosage form.

### Methods of Treatment

Another preferred aspect of the present subject matter pertains to a method of treating a skin disorder in a mammal, comprising topically administering to skin of a mammal in need thereof a therapeutically effective amount of a storage stable pharmaceutical composition providing improved tolerance upon topical administration comprising:

a) about 10 to about 45% by weight of an alpha hydroxy acid or a pharmaceutically acceptable salt thereof as an active ingredient;

b) a petroleum derivative;

c) a silicone containing emulsifier;

d) water; and

e) remaining amounts of one or more dermatologically acceptable excipients;

wherein said petroleum derivative and said silicone containing emulsifier are present in a ratio sufficient to render said composition well tolerated upon topical administration.

Several specific skin disorders may be treated according to these preferred methods. Exemplary among these skin disorders are cracked skin, dry skin, ichthyotic atopic eczema, xerosis, rough skin, dermatitis, psoriasis, ichthyosis, keratosis, keratoderma, corns, calluses, scales, age spots, wrinkles, warts, blemished skin, hyperpigmented skin, hyperkeratotic skin, inflammatory dermatoses, skin changes associated with aging, striae distensae, onychomycosis, acanthosis nigricans, acne vulgaris, child's syndrome, confluent and reticulated papillomatosis, ephelides (freckles), eruptive vellus hair cysts, ichthyosis vulgaris, keratoma, knuckle pads, lamellar ichthyosis, lentigo, Lichen spinulosus, liver spots, Netherton syndrome, Nevus comedonicus, photoaging, Pityriasis rotunda, Pseudoatrophoderma colli, Refsum disease, Rud's syndrome, steroid atrophy, Tinea pedis, and combinations thereof.

Included among the various kerotoses and keratoderma that may be treated according to the preferred methods are actinic keratosis, axillary granular parakeratosis, Erythrokeratoderma variabilis, palmaris and plantaris keratoderma, Keratosis pilaris, nipple/areolar hyperkeratosis, seborrheic keratoses, and combinations thereof.

In a particularly preferred embodiment, the present methods contemplate a method of treating keratosis pilaris in a mammal. Such methods are achieved by topically administering to skin of a mammal in need thereof a therapeutically effective amount of an improved tolerance, storage stable pharmaceutical composition suitable for topical administration comprising about 10 to about 45% by weight of an alpha hydroxy acid or a pharmaceutically acceptable salt thereof as an active ingredient.

In addition to treating these various skin disorders, the preferred methods also contemplate using the compositions described herein for moisturizing skin in a mammal. In this regard, the topical administration of preferred compositions to skin of a mammal can moisturize the skin or change keratinization of the skin. Similarly, the topical administration of preferred compositions to skin of a mammal can have such other, related effects on the skin as corneocyte hydration, a reduction in corneocyte adhesion, an increase in keratinocyte turnover, and an increase in fibroblast activity.

Combination Therapy

In another preferred embodiment, the present preferred compositions may be used in combination with an additional pharmaceutical dosage form to enhance their effectiveness in treating a dermatological disease or disorder. In this regard, the present preferred compositions may be administered as part of a regimen additionally including any other pharmaceutical and/or pharmaceutical dosage form known in the art as effective for the treatment of a dermatological disorder. Similarly, an active ingredient other than those specified herein can be added to the present preferred compositions to enhance their effectiveness in treating a dermatological disease or disorder. Accordingly, this additional active ingredient or additional pharmaceutical dosage form can be applied to a patient either directly or indirectly, and concomitantly or sequentially, with the preferred compositions described herein.

In one embodiment in this regard, the present preferred composition and the additional pharmaceutical dosage form can be administered to a patient at the same time. In an alternative embodiment, one of the present preferred compositions and the additional pharmaceutical dosage form can be administered in the morning and the other can be administered in the evening.

### Methods of Production

Another preferred aspect relates to a process for preparing a storage-stable pharmaceutical composition providing improved tolerance upon topical administration, said process comprising:
1) preparing an aqueous phase comprising about 10 to about 45% by weight of the overall weight of the composition of an alpha hydroxy acid or a pharmaceutically acceptable salt thereof as an active ingredient, about 0.005 to about 0.5% by weight of the overall weight of the composition of a chelating agent, and water at a temperature of about 55 to about 83° C.;
2) preparing an oil phase comprising a petroleum derivative and a silicone containing emulsifier in a weight ratio of about 0.75:1 to about 1:0.75, and about 0.02 to about 0.225% by weight of the overall weight of the composition of an antioxidant at a temperature of about 55 to about 85° C.;
3) adding said oil phase to said aqueous phase while mixing at a temperature of about 55 to about 83° C. to obtain a homogenous emulsion;
4) cooling said emulsion to a temperature of about 15 to about 30° C.; and
5) recovering a storage-stable, well tolerated topical pharmaceutical composition.

In another preferred embodiment, the aqueous phase is prepared by first mixing the active ingredient in purified water before adding the chelating agent. Similarly, the oil phase is preferably prepared by mixing the petroleum derivative, the silicone containing emulsifier, and the antioxidant at a temperature of about 55 to about 85° C. until melted. In a more preferred embodiment, the oil phase is prepared at a temperature of about 58 to about 75° C. In a most preferred embodiment, the oil phase is prepared at a temperature of about 60 to about 65° C.

In a further preferred embodiment, the oil phase is added to the aqueous phase while mixing for at least ten minutes. The cooling of the emulsion is preferably conducted in a stepwise manner, wherein the emulsion is cooled first to a temperature of about 42 to about 52° C., then to a temperature of about 34 to about 45° C., then to a temperature of about 28 to about 33° C., before finally being cooled to a temperature of about 22 to about 27° C. Additionally, before the emulsion is cooled to a temperature of about 34 to about 45° C., sufficient amounts of a buffering agent are added to provide the composition with a pH of about 2 to about 8.

This particular preparation process is a non-limiting example of a possible process that can be used to prepare preferred compositions. Other processes capable of preparing these compositions are further contemplated herein. Further, the individual phases of the preferred compositions (for example aqueous and oil phases) can be prepared sequentially in any order or concurrently; it is not necessary to prepare the aqueous phase before the oil phase is prepared in order to practice the present processes. Additionally, preferred compositions can be prepared according to either a batch process or continuously.

Further contemplated as within the scope of the present subject matter are pharmaceutical compositions produced according to the above-described process. If produced according to this process, these compositions exhibit chemical and physical stability suitable for topical administration.

The compositions produced according to these processes can further be used in a lotion, cream, ointment, shampoo, gel, aerosol, or other pharmaceutically acceptable topical dosage form. These compositions can be placed in a suitable containment vessel comprising a product contact surface composed of a material selected from the group consisting of glass, plastic, steel, stainless steel, aluminum, Teflon, polymeric structure, ceramic structure, alloys, and mixtures thereof. These containment vessels are used to facilitate manufacturing, handling, processing, packaging, storage, and administration of said composition.

### Dosage

Appropriate dosage levels for the alpha hydroxy acid active agent contemplated in the preferred compositions and methods are well known to those of ordinary skill in the art. Dosage levels on the order of about 0.001 mg to about 5,000 mg per kilogram body weight of the alpha hydroxy acid active therapeutic compound or compositions thereof are known to be useful in the treatment of the diseases, disorders, and conditions contemplated herein. Typically, this effective amount of the alpha hydroxy acid active agent will generally comprise from about 0.1 mg to about 100 mg per kilogram of patient body weight per day. Moreover, it will be understood that this dosage of active therapeutic agents can be administered in a single or multiple dosage units to provide the desired therapeutic effect.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

The preferred pharmaceutical compositions may be given in a single or multiple doses daily. In a preferred embodiment, the pharmaceutical compositions are given from one to three times daily. Starting with a low dose twice daily and slowly working up to higher doses if needed is a preferred strategy. The amount of active ingredients that may be combined with the carrier materials to produce a single dosage form will vary depending upon the host treated, the nature of the disease, disorder, or condition, and the nature of the active ingredients.

It is understood, however, that a specific dose level for any particular patient will depend upon a variety of factors well known in the art, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular drug or drug combination and the desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference in its entirety. Such formulations may influence the physical state, stability, rate of in vivo release, and rate of in vivo clearance of the therapeutic agents.

### EXAMPLES

The following examples are illustrative of preferred compositions and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates the preparation of a present preferred cream:

| | % W/W |
|---|---|
| Purified Water | 39.645 |
| Methylparaben | 0.075 |
| Citric Acid | 0.01 |
| Xanthan Gum | 0.30 |
| Light Mineral Oil | 7.00 |
| Cyclomethicone | 6.60 |
| Glycerin | 2.00 |
| Propylparaben | 0.03 |
| Butylated Hydroxytoluene | 0.05 |
| Steareth-2 | 3.30 |
| Steareth-21 | 1.70 |
| Cetostearyl Alcohol | 3.00 |
| Lactic Acid USP | 34.09 |
| Sodium Hydroxide | 2.20 |
| | 100.0% |

Preparation of the lotion:

1. An aqueous phase is prepared by mixing the Lactic Acid USP in purified water at a temperature of 73±2° C. The Glycerin, Edetate Disodium, and Methylparaben are then slowly added to this mixture successively one at a time and then mixed for 5 minutes. Temperature is maintained at 73±2° C. The Xanthan Gum is then sprinkled slowly on the surface of the mixture. A Sodium Hydroxide solution is then added to the mixture and mixed for 20 minutes.

2. An oil phase is prepared by mixing the Light Mineral Oil, the Propylparaben, the Butylated Hydroxytoluene, the Steareth-2, the Steareth-21, the Dimethicone, and the Cetostearyl Alcohol at a temperature of 73±2° C. until all ingredients have melted and a uniform blend results.

3. The oil phase is then slowly added to the aqueous phase at a temperature of 73±2° C. and mixed for 10 minutes to form an emulsion. The emulsion is then cooled to a temperature of 47±2° C. while mixing. The pH of the emulsion is then checked. If the pH is not between 2.8 and 3.2, a buffering agent is added and mixed in for 10 minutes to adjust the pH to the desired range. The emulsion is then cooled to a temperature of 40±2° C. while mixing. The emulsion is then further cooled to a temperature of 30±2° C. while mixing. The emulsion is then yet further cooled to a temperature of 25±2° C. while mixing.

### Example 2

The following example illustrates the preparation of an alternative preferred cream:

| | % W/W |
|---|---|
| Purified Water | 39.345 |
| Methylparaben | 0.075 |
| Phosphoric Acid | 0.01 |
| Lactic Acid USP | 34.09 |
| Edetate Disodium | 0.20 |
| Glycerin | 2.00 |
| Xanthan Gum | 0.30 |
| Sodium Hydroxide | 2.20 |
| Propylparaben | 0.03 |
| Butylated Hydroxytoluene | 0.05 |
| Light Mineral Oil | 7.00 |
| Vitamin E | 0.10 |
| Cyclomethicone | 6.60 |
| Cetostearyl Alcohol | 3.00 |
| Steareth-2 | 3.30 |
| Ceteareth-20 | 1.70 |
| | 100.0% |

This cream is prepared according to the procedure set forth above with respect to Example 1.

### Example 3

The following example illustrates the preparation of another alternative preferred cream:

| | % W/W |
|---|---|
| Purified Water | 39.545 |
| Methylparaben | 0.075 |
| Citric Acid | 0.01 |
| Xanthan Gum | 0.30 |
| Light Mineral Oil | 7.00 |
| Cyclomethicone | 6.60 |
| Glycerin | 2.00 |
| Propylparaben | 0.03 |
| Butylated Hydroxytoluene | 0.05 |
| Steareth-2 | 3.30 |
| Steareth-21 | 1.70 |
| Vitamin E | 0.10 |
| Cetostearyl Alcohol | 3.00 |
| Lactic Acid USP | 34.09 |
| Sodium Hydroxide | 2.20 |
| | 100.0% |

This cream is prepared according to the procedure set forth above with respect to Example 1.

### Example 4

A patient is suffering from keratosis pilaris. A preferred pharmaceutical composition herein is topically administered to the patient. It would be expected that the patient would improve his/her condition or recover.

### Example 5

A patient is suffering from dry skin. A preferred pharmaceutical composition herein is topically administered to the patient. It would be expected that the patient would improve his/her condition or recover.

### Example 6

A patient is suffering from eczema. A preferred pharmaceutical composition herein is topically administered to the patient. It would be expected that the patient would improve his/her condition or recover.

### Example 7

A patient is suffering from ichthyosis. A preferred pharmaceutical composition herein is topically administered to the patient. It would be expected that the patient would improve his/her condition or recover.

### Example 8

A patient is suffering from rough skin. A preferred pharmaceutical composition herein is topically administered to the patient. It would be expected that the patient would improve his/her condition or recover.

### Example 9

A patient is suffering from dermatitis. A preferred pharmaceutical composition herein is topically administered to the patient. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

We claim:

1. A storage-stable pharmaceutical composition providing improved tolerance upon topical administration comprising:
a) about 10 to about 45% by weight of an alpha hydroxy acid or a pharmaceutically acceptable salt thereof as an active ingredient;
b) a petroleum derivative;
c) a silicone containing emulsifier;
d) water; and
e) remaining amounts of one or more dermatologically acceptable excipients;
wherein said petroleum derivative and said silicone containing emulsifier are present in a ratio sufficient to render said composition storage-stable and well tolerated upon topical administration.

2. The pharmaceutical composition of claim 1, comprising about 30% by weight of pure alpha hydroxy acid in free acid form.

3. The pharmaceutical composition of claim 1, wherein said alpha hydroxy acid or pharmaceutically acceptable salt thereof is present in said composition as a mixture of an acid and a salt.

4. The pharmaceutical composition of claim 1, wherein said alpha hydroxy acid or pharmaceutically acceptable salt thereof is selected from the group consisting of atrolactic acid, benzilic acid, 4-chloromandelic acid, citric acid, 3,4-dihydroxymandelic acid, ethyl pyruvate, galacturonic acid, gluconolactone, glucuronic acid, glucuronolactone, glycolic acid, 2-hydroxybutanoic acid, 2-hydroxypentanoic acid, 2-hydroxyhexanoic acid, 2-hydroxyheptanoic acid, 2-hydroxyactanoic acid, 2-hydroxynonanoic acid, 2-hydroxydecanoic acid, 2-hydroxyundecanoic acid, 4-hydroxymandelic acid, 3-hydroxy-4-methoxymandelic acid, 4-hydroxy-3-methoxymandelic acid, α-hydroxyarachidonic acid, α-hydroxybutyric acid, α-hydroxyisobutyric acid, α-hydroxylauric acid, α-hydroxymyristic acid, α-hydroxypalmitic acid, α-hydroxystearic acid, 3-(2′-hydroxyphenyl)lactic acid, 3-(4′-hydroxyphenyl)lactic acid, lactic acid, malic acid, mandelic acid, methyllactic acid, methylpyruvate, mucic acid, α-phenylactic acid, α-phenylpyruvic acid, pyruvic acid, saccharic acid, tartaric acid, tartronic acid, pharmaceutically acceptable salts thereof, derivatives thereof, and mixtures thereof.

5. The pharmaceutical composition of claim 4, wherein said alpha hydroxy acid or pharmaceutically acceptable salt thereof is lactic acid or a pharmaceutically acceptable salt thereof.

6. The pharmaceutical composition of claim 1, wherein said petroleum derivative and said silicone containing emulsifier are present in said composition in a weight ratio of about 0.75:1 to about 1:0.75.

7. The pharmaceutical composition of claim 1, comprising about 3.75 to about 10% by weight of said petroleum derivative.

8. The pharmaceutical composition of claim 1, wherein said petroleum derivative is selected from the group consisting of a paraffin, a mineral oil, petrolatum, a synthetic or semi-synthetic hydrocarbon, derivatives thereof, and mixtures thereof.

9. The pharmaceutical composition of claim 1, comprising about 5 to about 8% by weight of said silicone containing emulsifier.

10. The pharmaceutical composition of claim 1, wherein said silicone containing emulsifier is a polysiloxane or a derivative thereof.

11. The pharmaceutical composition of claim 10, wherein said polysiloxane is selected from the group consisting of cyclomethicone, dimethicone, pharmaceutically acceptable salts thereof, derivatives thereof, and mixtures thereof.

12. The pharmaceutical composition of claim 1, further comprising up to 12% by weight of an additional emulsifier.

13. The pharmaceutical composition of claim 12, wherein said additional emulsifier is selected from the group consisting of straight or branched chain fatty acids, polyoxyethylene sorbitan fatty acid esters, lecithin, sorbitan fatty acid esters, propylene glycol stearate, glyceryl stearate, polyethylene glycol, fatty alcohols, ethoxylated fatty alcohol, fatty alcohol esters, fatty alcohol ethers, polymeric ethylene oxide-propylene oxide block polymers, derivatives thereof, pharmaceutically acceptable salts thereof, and mixtures thereof.

14. The pharmaceutical composition of claim 13, wherein said additional emulsifier is selected from the group consisting of steareth-2, steareth-21, ceteareth-20, cetostearyl alcohol, derivatives thereof, pharmaceutically acceptable salts thereof, and mixtures thereof.

15. The pharmaceutical composition of claim 1, wherein said dermatologically acceptable excipients are selected from the group consisting of surfactants, preservatives, emollients, humectants, fluid alkyl alcohols, thickening agents, suspending agents, pH modifiers, chelating agents, antioxidants, derivatives thereof, and mixtures thereof.

16. The pharmaceutical composition of claim 1, wherein said composition contains a sufficient amount of a buffering agent to provide an overall pH of about 2 to about 8 to said composition.

17. The pharmaceutical composition of claim 1, wherein said composition is in a lotion, cream, ointment, shampoo, gel, aerosol, or other pharmaceutically acceptable topical dosage form.

18. A method of treating a skin disorder in a mammal, comprising topically administering to skin of a mammal in need thereof a therapeutically effective amount of a storage stable pharmaceutical composition providing improved tolerance upon topical administration comprising:
a) about 10 to about 45% by weight of an alpha hydroxy acid or a pharmaceutically acceptable salt thereof as an active ingredient;
b) a petroleum derivative;
c) a silicone containing emulsifier;
d) water; and
e) remaining amounts of one or more dermatologically acceptable excipients;
wherein said petroleum derivative and said silicone containing emulsifier are present in a ratio sufficient to render said composition well tolerated upon topical administration.

19. The method of claim 18, wherein said skin disorder is selected from the group consisting of cracked skin, dry skin, ichthyotic atopic eczema, xerosis, rough skin, dermatitis, psoriasis, ichthyosis, keratosis, keratoderma, corns, calluses, scales, age spots, wrinkles, warts, blemished skin, hyperpigmented skin, hyperkeratotic skin, inflammatory dermatoses, skin changes associated with aging, striae distensae, onychomycosis, acanthosis nigricans, acne vulgaris, child's syndrome, confluent and reticulated papillomatosis, ephelides (freckles), eruptive vellus hair cysts, ichthyosis vulgaris, keratoma, knuckle pads, lamellar ichthyosis, lentigo, Lichen spinulosus, liver spots, Netherton syndrome, Nevus comedonicus, photoaging, Pityriasis rotunda, Pseudoatrophoderma colli, Refsum disease, Rud's syndrome, steroid atrophy, Tinea pedis, and combinations thereof.

20. The method of claim 19, wherein said kerotosis or keratoderma is selected from the group consisting of actinic keratosis, axillary granular parakeratosis, Erythrokeratoderma variabilis, palmaris and plantaris keratoderma, Keratosis pilaris, nipple/areolar hyperkeratosis, seborrheic keratoses, and combinations thereof.

21. The method of claim 18, wherein said pharmaceutical composition is administered concomitantly or sequentially with an additional pharmaceutical dosage form effective to treat said skin disorder.

22. A method of treating keratosis pilaris in a mammal, comprising topically administering to skin of a mammal in need thereof a therapeutically effective amount of an improved tolerance, storage-stable pharmaceutical composition suitable for topical administration comprising about 10 to about 45% by weight of an alpha hydroxy acid or a pharmaceutically acceptable salt thereof as an active ingredient.

23. The method of claim 22, wherein said pharmaceutical composition further comprises:
a) a petroleum derivative;
b) a silicone containing emulsifier;
c) water; and
d) remaining amounts of one or more dermatologically acceptable excipients;
wherein said petroleum derivative and said silicone containing emulsifier are present in a ratio sufficient to render said composition well tolerated upon topical administration.

24. The method of claim 22, wherein said topical administration moisturizes said skin or changes keratinization of said skin.
